(19) United States
(12) Patent Application Publication
(10) Pub. No.: US 2009/0171268 A1
(43) Pub. Date: Jul. 2, 2009

(54) Manually Operated Insufflator

(76) Inventors: Robert C. Williams, JR., Fort Salonga, NY (US); Harry Bratton, Lajolla, CA (US); Brad S. Schreck, Totowa, NJ (US); Vito De Monte, North Babylon, NY (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/568,153

(22) PCT Filed: Apr. 21, 2005
(86) PCT No.: PCT/US05/13825

§ 371 Date: Nov. 7, 2008

Related U.S. Application Data
(60) Provisional application No. 60/564,706, filed on Apr. 23, 2004.

(51) Int. Cl. A61M 13/00

(52) U.S. Cl. 604/26

(57) ABSTRACT

The invention includes a device, kit, and system capable of manually distending a subject's body cavity. In one embodiment, the invention includes a distention media reservoir (10), insertable member (20) for insertion into a body cavity, and a manually operated pumping member (12) capable of displacing distention media from the reservoir and into a body cavity. In some embodiments, the distention media reservoir may be air or CO₂. In one embodiment, the invention is available in the form of a kit. In another embodiment, the devices include a reusable portion and a consumable portion. The reusable portion may include the media distention reservoir and pumping member, the consumable portion includes the insertable member, and may also include a barrier for preventing effluent from contaminating the reusable. In other embodiments, the device is adapted to function as a distention media booster pump that may be used in conjunction with an electro-pneumatic insufflation system.

### BACKGROUND OF THE INVENTION

The invention relates generally to devices for distending the body cavity of a subject, and more particularly to a device for manually distending a body cavity.

Colonoscopy is a medical procedure whereby a physician can view the inside of the large intestine, from the lowest part, the rectum, all the way up through the colon to the lower end of the small intestine. The procedure may be used to look for early signs of cancer in the colon and rectum. It may also be used to diagnose the causes of unexplained changes in bowel habits. During a colonoscopy a physician may be able to see inflamed tissue, abnormal growths, ulcers, bleeding, and the like.

Visual and virtual colonoscopies are two common techniques of performing a colonoscopy. In a visual colonoscopy, a colonoscope, is inserted through the rectum and into the large intestine. A colonoscope is a long, thin, flexible tube with imaging optics and a light source on the end. By adjusting the various controls on the colonoscope, the physician can carefully guide the instrument in any direction to look at the inside of the colon. The images are displayed on a TV monitor or similar display. Patients undergoing a visual colonoscopy are typically sedated during the procedure.

Virtual colonoscopy is a relatively new technique that uses computed tomography (CT) scanner and computer virtual reality software to look inside the body without having to insert a colonscope into the colon or sedate the patient. The scanner takes about 30 seconds to complete its scan. The data may then be reconstructed by a computer into either 2-dimensional sectional images or into a 3-dimensional rendered image of the colon that can be reviewed from all angles by a physician.

During the virtual colonoscopy procedure, air may be introduced into the colon to inflate or distend the colon. Ideally, distention may be maintained throughout the procedure to obtain the most accurate image. Currently, it is common to distend the colon or other body parts of a subject prior to and during examination by direct connection of an insufflator to the proximal end of a rectal catheter inserted into the rectum of the subject. With this device, a distention media such as air or CO₂, for example is introduced into the colon. Conventional insufflator devices may use an electro-pneumatic pump to introduce a distending fluid or media into the colon. The use of automatic insufflation devices can sometimes be cost prohibitive for practitioners who infrequently perform virtual colonoscopy by adding the expense associated with purchasing and maintaining an electromechanical piece of diagnostic equipment. In addition, monitoring and controlling fluid flow from the insufflator to the colon may also add to the overall complexity of the procedure for practitioners who perform this procedure infrequently.

Thus, there still exists a need for manual insufflator devices that are less expensive and less complex.

### BRIEF SUMMARY OF THE INVENTION

The present invention encompasses an insufflator device, system, kit, and method that may be used to manually introduce a distention media into the colon or other body cavity. The invention may provide a cost-effective alternative to electro-pneumatic powered insufflator devices. As a result, the invention may be particularly useful for distending the body cavity of an individual in diagnostic medical procedures, such as virtual colonoscopy.

In one alternative embodiment, the invention comprises a manually operable pumping member adapted for introducing a distention media from a distention media reservoir, through an insertable member, such as a rectal catheter, and into the body cavity of a subject. In some embodiments the pumping member may be disposed between the distention media reservoir and the insertable member. The pumping member may comprise a variety of different manually operable devices that can be used to pump a fluid, such as gas. In one alternative embodiment, the pumping member may comprise a hand bulb having a hollow shaped structure, such as a bulb, ball, ellipsoidal, or other spherical shaped structure. The pumping member may be sized to fit within the palm of a person's hand so that pumping pressure can easily be applied with the use of one hand.

The distention media reservoir may comprise a source of fluid, such as air or CO₂ that may be used to distend a desired body cavity. In one embodiment, the reservoir may comprise a flexible pouch having a gas such as CO₂ disposed therein. In some embodiments the reservoir can be filled with gas by filling it from a pressurized tank, external canister, disposable CO₂ cartridge, or the gas may be a byproduct of a chemical reaction. In still other embodiments, the reservoir may comprise ambient air that is manually displaced into a body cavity directly from the surrounding atmosphere.

In some embodiments, the manually operable insufflator device may be used with a conventional electro-pneumatic insufflator. In this embodiment, the pumping member may provide a means to more precisely control and optimize the volume of gas being introduced into a body cavity. The manually operable insufflator portion may also include a flexible pouch that can be filled with CO₂ to serve as an in-line reservoir. Alternatively in some embodiments, the pumping member may displace air directly from the surrounding atmosphere into the subject's body cavity.

In one alternate embodiment, the insufflator device may be in the form of a self-contained kit having the necessary elements prepared for assembly at the point of use. In some embodiments the insufflator device may also include an in-line effluent trap for trapping effluent. The effluent trap may be disposed between the inserting member and the pumping member. In yet another embodiment, the elements of the kit may be sterile, reusable, and/or disposable.

In yet another alternate embodiment, the insufflator device may comprise a reusable portion and a consumable portion that may be discarded after use. In this embodiment, the reservoir, pumping member, and connecting tubing may comprise a reusable portion that may be repeatedly reused. In this embodiment, the reservoir may comprise the ambient atmosphere or a collapsible reservoir that can be filled with CO₂ from an external canister, disposable CO₂ cartridge, and the like. The consumable portion may comprise the inserting member and a filter barrier to prevent effluent from the body cavity from entering and contaminating the reusable portion. The consumable portion may also include a new CO₂ cartridge. An interlocking fitting may be used to connect the reusable portion to the consumable portion.

In one alternative embodiment, the present invention may provide a lower-cost and efficient means for introducing a distention media into a body cavity of a subject. Other features of the present invention are set forth in the drawings and detailed description.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is a non-limiting description of one alternative embodiment of the present invention showing a graphical illustration of a consumable manually operated insufflator device;

FIG. 2 is a non-limiting description of one alternative embodiment of the present invention showing a graphical illustration of an insertable member that may be used in the practice of the invention;

FIGS. 3a through 3c are non-limiting descriptions of one alternative embodiment of the present invention showing graphical illustrations of various insertable members depicting alternative retaining means;

FIG. 4a is a non-limiting description of one alternative embodiment of the present invention showing a graphical illustration of a manually operated insufflator device and/or kit having a reusable portion and consumable portion, and wherein the reusable portion includes a distention media reservoir;

FIG. 4b is a non-limiting description of one alternative embodiment of the present invention showing a graphical illustration of a clamp that may be used with the manual insufflator device;

FIG. 5 is a non-limiting description of one alternative embodiment of the present invention showing a graphical illustration of filling a distention media reservoir via an external canister;

FIG. 6a is a non-limiting description of one alternative embodiment of the present invention showing a graphical illustration of an assembled manual insufflator device that may be available as a kit;

FIG. 6b is a non-limiting description of one alternative embodiment of the present invention showing a graphical illustration of the manual insufflator device depicted in FIG. 6a in a packaged and unassembled state. In one alternative embodiment, the packaged items are sterile, reusable, and/or disposable;

FIG. 7 is a non-limiting description of one alternative embodiment of the present invention showing a graphical illustration of a consumable manual insufflator device that may be adapted to draw distention media from both an attached distention media reservoir and the surrounding atmosphere;

FIG. 8 is a non-limiting description of one alternative embodiment of the present invention showing a graphical illustration of a manual insufflator device having a reusable portion and a consumable portion, and wherein a CO₂ cartridge may be used to fill the reservoir with CO₂ gas;

FIGS. 9a and 9b are non-limiting descriptions of one alternative embodiment of the present invention showing graphical illustrations of alternative manual insufflator devices that have both a reusable portion and a consumable portion, and wherein each draws air as the distention media directly from the surrounding atmosphere;

FIG. 10 is a non-limiting description of one alternative embodiment of the present invention showing a graphical illustration of a manual insufflator device that has a reusable portion and a consumable portion;

FIG. 11 is a non-limiting description of one alternative embodiment of the present invention showing a graphical illustration of an insufflator system comprising an electro-pneumatic insufflation unit and a manual insufflator device;

FIG. 12 is a non-limiting description of one alternative embodiment of the present invention showing a graphical illustration of the manual insufflator device depicted in FIG. 11; and

FIG. 13 is a non-limiting description of one alternative embodiment of the present invention showing a graphical illustration of an alternative manual insufflator device that may be adapted for use with an electro-pneumatic insufflation unit and draws air as the distention media directly from the surrounding atmosphere.

### DETAILED DESCRIPTION OF THE INVENTION

The present invention now will be described hereinafter with reference to the accompanying drawings. The invention may be embodied in many different forms and the drawings and descriptions herein should not be construed as limited to the embodiments set forth herein. Like numbers refer to like elements throughout.

In one alternative embodiment, the invention encompasses an insufflator device, system, kit, and method for the manual distention of a subject's body cavity. More specifically, the invention may comprise a device adapted for manually introducing a distention media, such as, for example, CO₂ or air, into the body cavity of a subject in connection with performing a medical or diagnostic procedure of an anatomical area of interest. Distention media may include liquid, gas, or solid media. Such procedures may include, but are not limited to, gastrointestinal imaging, including, but not limited to X-ray imaging, or virtual gastrointestinal imaging, for example. Virtual gastrointestinal imaging includes any technique of using computer software to view the inside of any section of the gastrointestinal tract, including CT imaging, MR imaging, PET imaging, or the like. Such medical diagnostic procedures may also include fiberoptic, endoscopy, colonoscopy, sigmoidoscopy, and the like, and combinations thereof.

In one alternative embodiment, the invention comprises a pumping member that may be disposed between a distention media reservoir and an insertable member. The pumping member may be manually operated to pump distention media from the distention media reservoir through the insertable member and into the body cavity of a subject. It should be recognized that the invention is not limited to performing medical procedures on human patients, but that the invention can also be used in conjunction with medical procedures performed on animals.

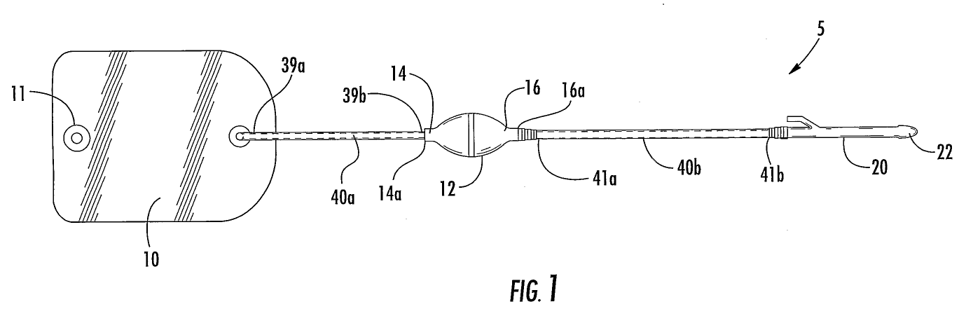

Referring to the figures, FIG. 1 illustrates an alternative embodiment of the insufflator device that is broadly designated as reference number 5. As shown, insufflator device 5 may comprise a distention media reservoir 10, a pumping member 12, and an insertable member 20. The insufflator device may also include multiple fluid conduits 40a, 40b, collectively referred to as reference number 40, for providing a fluid pathway from the distention media reservoir 10 through the insertable member 20, and into a desired body cavity. During operation, the tip 22 of the insertable member may be placed into the entrance of a body cavity, such as the anus. An operator may then apply pumping pressure to the pumping member to displace the distention media from the reservoir and introduce it into the body cavity.

In some embodiments, a first flow conduit 40a defines a fluid pathway between the pumping member 12 and the distention media reservoir 10, and a second conduit 40b defines a fluid pathway between the pumping member 12 and the insertable member 20. As shown, the distal end 39a of the first conduit 40a may be interfaced with the distention media reservoir, and the proximal end 39b may be interfaced with an inlet 14a on the pumping member 12. The second conduit 40b comprises a distal end 41a interfaced with an outlet 16a on the pumping member 12, and the proximal end 41b is interfaced with the insertable member.

The pumping member 12 may comprise a means by which distention media may be manually displaced from the distention media reservoir and introduced into a subject's body cavity. The pumping member means may comprise a variety of different devices and methods. Such means include, but are not limited to, a hand bulb or puffer, hand operated piston pump, bellows pump, and equivalents thereof. Exemplary pumping members include, without limitation, E-Z-EM hand bulb or E-Z-EM E-Z-Flat device, sold by E-Z-EM, Inc., of Westbury, N.Y.

In some embodiments, the pumping member comprises a hollow bulb, ball, ellipsoidal, or spherical shaped structure that may be adapted for confining and releasing a fluid upon the application of manual pressure. In some embodiments, the pumping member may comprise a flexible elastomeric material such as synthetic or natural rubber, soft plastic, polyurethane, latex, nylon, polytetrafluoroethylene (PTFE), silicones, or blends thereof. In some embodiments, the pumping member may be in the form of a hand bulb having two diametrically opposed nipple shaped orifices 14, 16 extending outwardly from the exterior surface. A check valve may be disposed within each of the orifices. Typically one of the check valves is biased to prevent flow into the bulb, and the other biased to prevent flow out of the bulb. Upon applying manual pressure to the bulb, fluid confined within the bulb exits the bulb through the check valve 16 biased to block air inflow. Upon release of manual pressure, fluid enters the bulb through the check valve 14, which is biased to prevent its flow out of the bulb. In some embodiments, the pumping member comprises an inlet 14a and an outlet 16a. Inlet 14a is adapted for providing a entrance for receiving distention media into the interior of the pumping member, and outlet 16a may be adapted for providing an exit through which distention media may be expelled from the interior of the pumping member. A relief or patient venting valve 18 may be disposed adjacent to the exit of the pumping member. Valve 18 may provide the ability to selectively or automatically relieve pressure or vent distention media from the cavity being inflated in the case it may be necessary to do so during the course of a medical procedure. Valve designs for this application may include, but are not limited, to rotary open-close, spring loaded open-close, or needle type. Alternatively, a pressure relief type valve may be adapted singularly or in combination with an open-close valve. Such a pressure relief type may have a fixed or adjustable relief pressure to automatically relieve inflated cavity pressure if it rises above a pressure relief valve set point.

In one alternative embodiment, the insertable member 20 comprises an apparatus that may be suitable for insertion into an opening of a cavity of a subject. The insertable member may have one or more hollow areas, such as a multilumen tube, for example. Possible body cavities include, but are not limited to, the mouth, vagina, uretha, ear, nostril, uterus, appendix, cecum, hepatitis flexure, splenic flexure, transverse colon, descending colon, sigmoid, rectum, sphincter, or any other body orifice, channel, or opening to a subject's body, including incisions into the subject's body. The insertable member may include, but is not limited to, an instrument for examining the interior of the subject's cavity such as a trocar, endoscope, enema tip, Foley catheter, entry needle, and the like, and combinations thereof. It may also include an instrument for administering a powder, gas, liquid, or vapor into a body cavity. The insertable member may also include an instrument or device for removing liquid, gas, or solid from the interior of a subject's cavity.

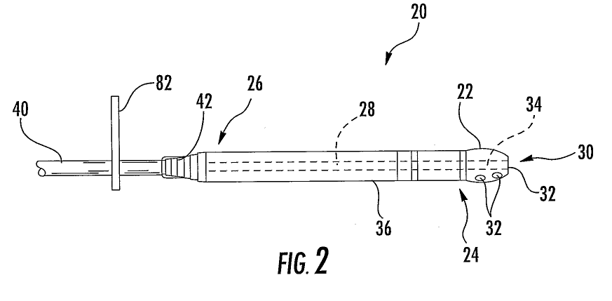

With reference to FIG. 2, the insertable member 20 may comprise a front portion 24 and a rear portion 26 having one or more connection means. Such connection means include, without limitation, means for forming a connection with one or more other components. In some embodiments the connection means include, but are not limited to, means for forming a Luer connection, Colder Products® connection, barbed connection, male/female type connection or any equivalent thereof. In the context of the invention, a Colder Products® connection refers to a fitting or coupling that may be connect, disconnected, and reconnected as needed. As shown, the rear portion of the insertable member may be connected to conduit 40 at 42. In some embodiments, the rear portion 26 of the insertable member may be interfaced with the proximal end 41b of conduit 40b (see FIG. 1).

The front portion 24 of the insertable member comprises a tip structure 22 supported thereon. The tip structure may be adapted to initiate entry of the insertable member into an opening to a subject's cavity. The insertable member may be structured so that part or at least substantially all of the insertable member is inserted through the opening of the subject's cavity. Thus, once inserted, the insertable member may be capable of maintaining an opening to the cavity. For insertion into the cavity, the tip may be lubricated so that it passes gently into the cavity. The tip can be removed from the cavity at any time by gentle traction therefrom.

In one embodiment, the insertable member 20 may have a hollow portion or channel 28 positioned internally. The hollow portion 28 extending from the front portion 24 of the insertable member to its rear portion 26. The tip structure may also comprise an apex 30. In some embodiments, the apex 30 of the tip structure 22 may have one or more openings 32 through which a fluid may pass. The one or more openings 32 may be interfaced with at least one hollow area 34 disposed in the interior of the tip structure 22. The tip structure 22 may comprise multiple openings 32 that are each interlaced with a hollow area 34 disposed inside the tip structure.

The configuration of the tip structure may comprise various shapes and forms. For example, the tip structure may be cylindrical or non-cylindrical. In one alternative embodiment, the circumference of the tip structure may be substantially equal to or greater than other portions of the insertable member. The shape or form of one or more sections of the tip structure may include, but is not limited to, annular, planar, circular, rounded concave, convex, conical, elliptical, ellipsoidal, conidial, crescent-like, helical, oblong, oval, parabolic, round, sinusoidal, spherical, hemi-spherical, tapered, tubular, triangular, wedge-like, head-like, or any other configuration capable of insertion into the opening of a subject's body cavity.

In another alternative embodiment, the insertable member may comprise a shaft 36 having a distal and proximal end. The tip structure 22 may be positioned adjacent to the proximal end of the shaft. The distal end may be interfaced with the pumping member, fluid conduit, or other member. The interior of the shaft 36 may comprise one or more hollow areas 28 extending along part of or at least substantially the entire length of the shaft. The hollow area of the shaft 28 can be completely or partially aligned with a hollow area 34 of the tip structure of the insertable member, thereby forming a conduit extending from the proximal end of the insertable member to its distal end. The tip structure 22 and shaft 36 may represent separate identifiable components or they may represent one single component of the insertable member. The insertable member 20 may comprise means 42 for fastening said member to one or more conduits 40 leading to the pumping member 12, or medical apparatus or any component thereof.

In some embodiments, the insertable member may comprise a solid, substantially rigid material. Such materials may also include polyvinylchloride (PVC) or polyethylene, for example. The insertable member may also comprise a substantially resilient material such as rubber or an elastomeric polymer, such as soft plastic, polyurethane, latex, nylon, polytetrafluoroethylene (PTFE), silicones, or blends thereof.

In some embodiments, the insertable member may be insertable into a subject's rectum. In one embodiment, the insertable member comprises a single lumen, Catalog No. 8816, E-Z-EM Flexi Tip®. This product is available from E-Z-EM of Westbury, N.Y. In another alternative embodiment, the tip structure may comprise a dip molded vinyl tip. The molded tip may be integrally connected to a locking pinch clamp.

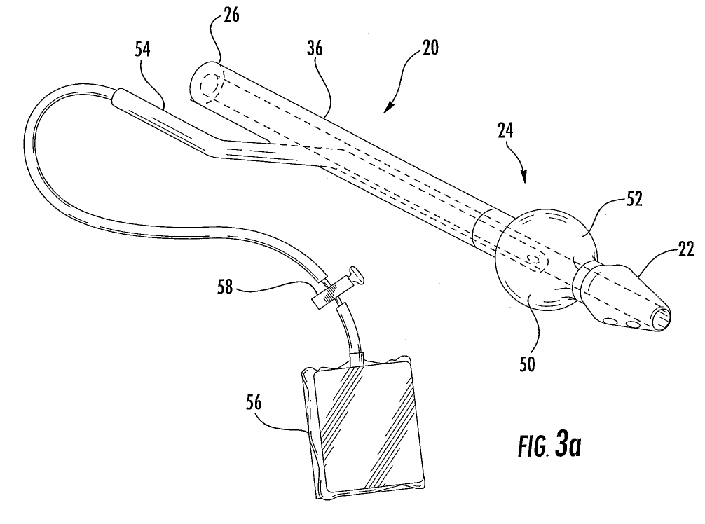

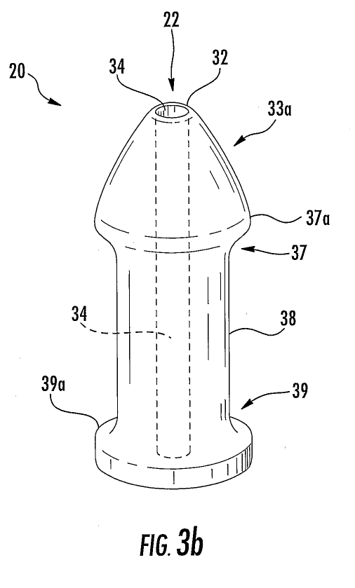

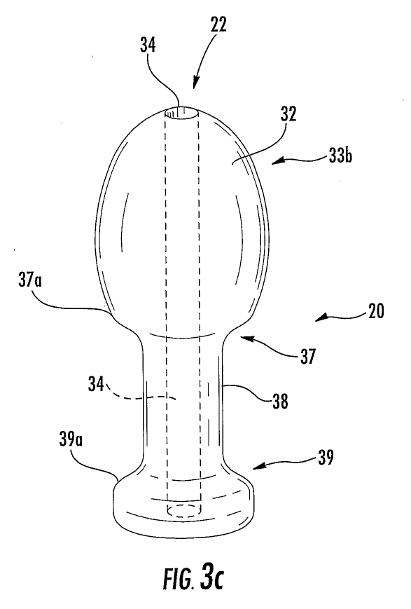

In some embodiments, the insertable member may also comprise one or more retaining means to prevent the insertable member from shifting positions after it has been inserted into a cavity. In this regard, FIGS. 3a through 3c illustrates various retaining means. FIG. 3a illustrates a retaining means comprising an expandable structure in one embodiment, the shaft 36 or tip structure 22 may include an expandable structure 50. In one alternative embodiment, the expandable structure 50 may take the form of a seal forming structure, such as, for example, an inflatable balloon-like structure 52 that can be supported on the shaft or tip structure 22 of the insertable member 20. The balloon like structure 52 may be connected to an inflation conduit 54 that may be positioned in the interior or exterior of the insertable member 20. The inflation conduit may be separate and independent of the interior lumen of the insertable member. The inflation conduit 54 may be interfaced and in fluid communication with an inflation pump 56 so that a gas or liquid may be pumped through the conduit 54 and into the balloon-like structure 52. Additionally, the inflation conduit may be interfaced with a stopcock, valve, or any other means for preventing or allowing the flow of liquid or gas from escaping or passing through the inflation conduit. In one embodiment, conduit may comprise an inflation control stopcock 58 and a means for attaching the inflation conduit to the nozzle of an inflation pump. In one embodiment, the expandable structure is an E-Z-EM Flexi Cuff® silicone elastomer retention cuff, or a similar device. This product is also available from E-Z-EM, Inc., of Westbury, N.Y.

When the insertable member 20 is inserted through the opening of a body cavity, the balloon-like structure 52 supported on the shaft 36 or the tip structure 22 can be inflated from its normal flat state into a distended balloon-like donut 52 to prevent undesirable movement of the insertable member 20.

In some embodiments, the insertable member 20 may also include an abutment position on or in the vicinity of the insertable member to prevent unwanted movement. In this regard, FIGS. 3b and 3c illustrate embodiments of the invention wherein the insertable member 20 may include a proximal portion 37, a center portion 38, and a distal portion 39. FIG. 3b illustrates a retaining means comprising a cone-shaped 33a tip structure, and FIG. 3c illustrates a retaining means comprising a cylinder head 33b having a first circumference along an axis. As shown, the proximal portion may include a tip structure 22 having a first radial abutment 37a at its base. The distal portion may have a diameter equal to or greater than the proximal portion, thus forming a second radial abutment surface 39a. The center portion 38 may comprise the shaft of the insertable member and has a diameter that is less than both the proximal and distal portion. The tip may be used to initiate entry of the insertable member into the body cavity. Once inserted, the orifice, or periphery thereof, of the body cavity is positioned between the first and second radial abutment surfaces (37a, 39a) to secure the insertable member inside the subject's body cavity.

In one alternative embodiment, the invention may also comprise a distention media reservoir. In some embodiments, the distention media reservoir may be in fluid communication with the insertable member. In other embodiments, the manual insufflator device may comprise a kit wherein the reservoir may be adapted to be in fluid communication with the insertable member upon assembling the insufflator device. In one alternative embodiment, the distention media reservoir may comprise a hollow interior capable of receiving and releasably confining distention media therein. In another alternative embodiment, the reservoir may comprise the atmosphere from which air can be directly drawn by pumping member.

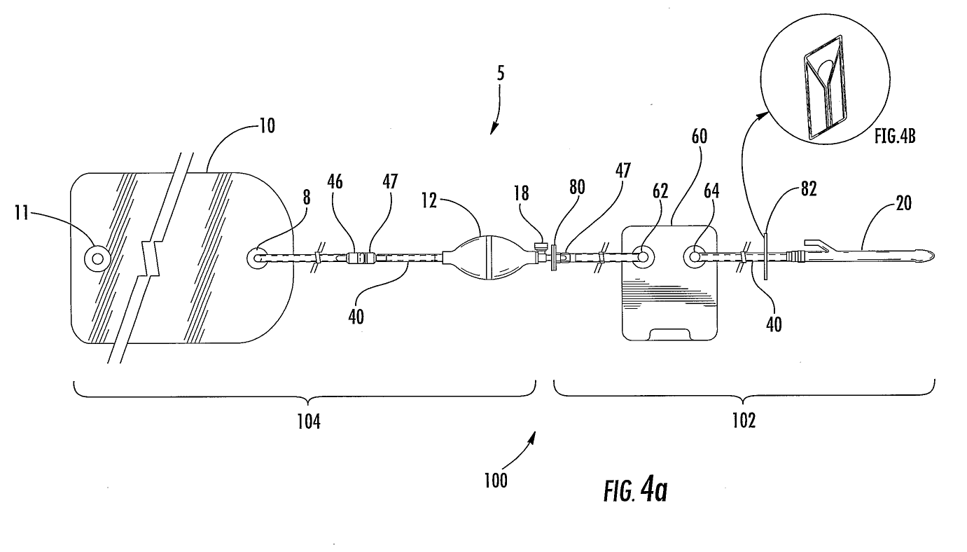

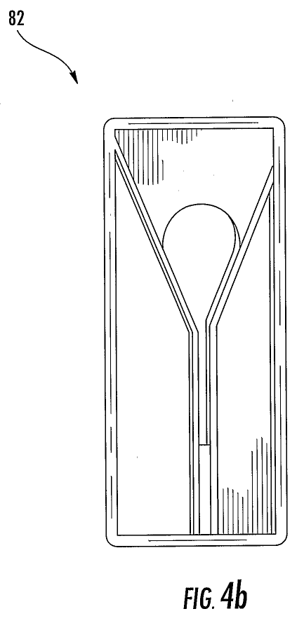

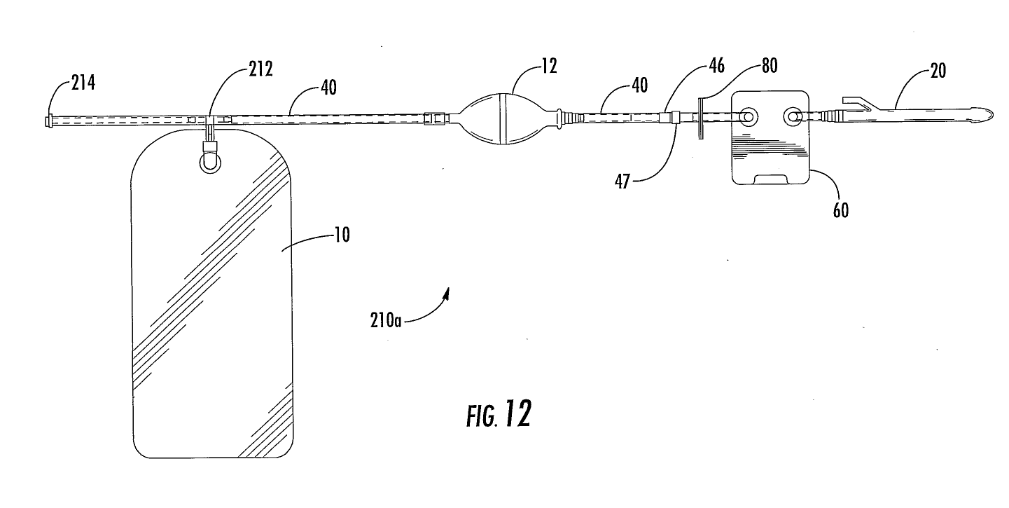

In some embodiments, the distention media reservoir comprises an interior area having a closed bottom, and front and rear walls secured together around their periphery. The reservoir may also comprise one or more ports or openings through which distention media can enter or exit the reservoir. The reservoir may also comprise one or more ports or openings through which distention media can be supplied for subsequent introduction into a subject's body cavity. Design of the distention media reservoirs in some embodiments of invention may best be facilitated by a deformable or collapsible container. In this regard, FIG. 4 illustrates an alternative embodiment of the distention media reservoir having access outlet 8 adapted for fluid communication with the insertable member. In some alternative embodiments, access outlet 8 may be adapted for removably receiving conduit 40. In some of these embodiments the access outlet 8 may comprise a grommet having a sealing plug through which the conduit can be inserted. The access outlet 8 may comprise connections, such as Luer connection, Colder Products® connection, barbed connection, male/female connection or any equivalent thereof. In other alternative embodiments, the conduit 40 may be permanently secured to the access outlet 8. In some embodiments, the access outlet 8 may be interfaced directly to the distal end 39a of flow conduit 40a (see FIG. 1). In other embodiments, the access outlet 8 may be interfaced to the conduit 40 via a 3-way connector, such as a T-connector (see FIG. 12, reference number 212). In another embodiment, the distention media reservoir 10 may also include a sealably openable access inlet 11 for receiving distention media. In some embodiments, the distention media reservoir 10 may be releasably attached to flow conduit 40 with male/female connectors (46, 47). If the reservoir 10 is depleted, a fresh reservoir can be attached by detaching the depleted reservoir and attaching the new reservoir. The distention reservoir may typically hold about 0.5 Liter to 20 Liter of distention media, preferably about 2 Liter to 6 Liter, more preferably about 3 Liter to 4 Liter.

In one embodiment, the distention media reservoir may comprise a container or pouch having a bag-like shape. Alternatively, the reservoir may have a bottle-like, tray-like, box-like, or tube-like shape, for example. One advantage of a collapsible container is its smaller material volume which may facilitate handling during manufacture, storage, shipping, use, and disposal.

The reservoir of the present invention may be prepared from a variety of suitable plastic materials whereby a strong, lightweight, reliable, yet economic container is provided. For example, in some embodiments the distention media reservoir may comprise a suitable elastomeric material, such as olefin-based materials, including but not limited to, polyethylene, ethylene-propylene copolymers, ethylene-vinyl acetate copolymers, ethylene-acrylic ester copolymers, iononomers, and combinations thereof. Additionally, film layers comprising polymers having gas barrier properties, such as polyvinylidene chloride and ethylene-vinyl alcohol copolymers, as well as film layers of such polymers as polyvinyl chloride, polyester, polyamide, and polyurethanes may also be used.

The distention media reservoir may also comprise any flexible material, including, polyethylene film, plasticized polyvinyl chloride film, plasticized polyvinylidene chloride film, polyethylene/ethylene-vinyl acetate copolymer laminate, ethylene-vinyl acetate copolymer/polyvinylidene chloride/ethylene-vinyl acetate copolymer laminate, and polyethylene/ethylene-vinyl acetate copolymer/polyethylene chloride/ethylene-vinyl acetate copolymer/polyethylene laminate, and combinations thereof.

The distention media may be selected from a variety of different materials that can be used to efficiently and safely distend a body cavity. Typically, the distention media comprises a fluid, such as a gas, that can be safely introduced into a subject's body cavity and then may be able to exit under normal physiological pathways. In an alternative embodiment, the distention media comprises CO₂ gas. CO₂ gas can be supplied to the reservoir from a variety of different sources including, but not limited to, a conventional CO₂ insufflator device, external CO₂ canisters including large canisters such as those commonly used in fire extinguishers, and small canisters such as those commonly used in paint ball guns, CO₂ cartridges, CO₂ gas formed from the sublimation of dry ice, and CO₂ gas that may be released as byproduct of a chemical reaction. In an alternative embodiment, the distention media may comprise air which may be extracted directly from the surrounding atmosphere.

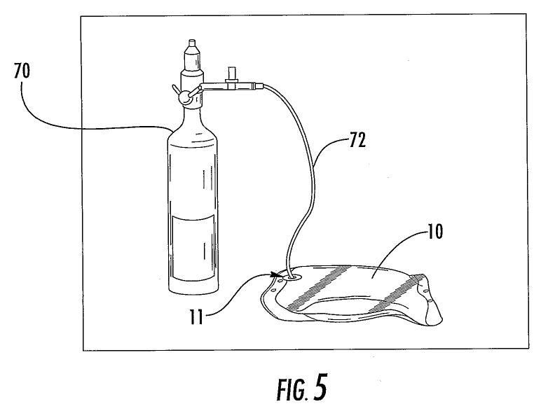

As shown in FIG. 4a, the distention media reservoir may comprise an access inlet 11 for supplying distention media into the reservoir. In some embodiments, the reservoir may be filled by inserting a tube that is connected to a pressurized canister comprising a suitable distention media. In this regard, FIG. 5 illustrates a distention media reservoir 10 in the process of being filled with CO₂ from an external canister 70. As shown, canister supply tube 72 may be inserted into the distention media reservoir through inlet 11. The distention media reservoir can then be filled with a desired volume of distention media. In another embodiment, a compound capable of producing a fluid may be inserted into the distention media reservoir, such as effervescent granules that release CO₂ gas upon treatment with water. For example, effervescent granules comprising sodium bicarbonate and citric acid can be placed into the media distention reservoir and mixed with a small amount of water, typically about 100 cc. Contact of the water with the granules produces CO₂ as a byproduct. The amount of granules inserted into the distention media reservoir may be controlled so that the reservoir is filled with a desired volume of gas.

As shown in the figures, the manual insufflator device may also include one or more conduits 40 defining a fluid pathway from the reservoir to the insertable member. In some embodiments, the fluid pathway may comprise flexible tubing that may be formed of a vinyl or equivalent plastic. The invention may also comprise various connectivity means for sealably connecting the various members and conduits to each other, and thereby define a fluid pathway between the distention media reservoir and the body cavity of the subject. Such connectivity means include, without limitation, Luer connection, Colder Products® connection, barbed connection, male/female connection, frictional fit connection, or any equivalent thereof. The male/female sections of these connections are collectively referred to by reference numbers 46 and 47, respectively. In one alternative embodiment, the connectivity means provides a means for forming a fluid-type seal between one or more portions of the conduits and the various members, including, but not limited to the distention media reservoir, pumping member, insertable member, and to one or more apparatuses used in the medical or diagnostic procedure. In some embodiments, the conduits 40 may be permanently fixed to the various components, such as the distention media reservoir, pumping member, or insertable member. In other embodiments, the connections may be reusable, for example, Luer, Colder Products®, etc.

In some embodiments, the manual insufflator device may also include an effluent collection reservoir for collecting any effluent expelled from a subject's body cavity during or after a medical or diagnostic procedure. The effluent collection reservoir prevents the effluent from impeding the administration of the distention media. In this regard, FIG. 4 illustrates an effluent collection reservoir 60 disposed between the pumping member 12 and the insertable member 20. As shown, the effluent collection reservoir includes openings 62, 64 through which distention media may flow as it travels from the distention media reservoir to the insertable member. The structure and use of suitable effluent collection reservoirs are described in commonly owned PCT Publication No. WO 03/045303, the disclosure of which is hereby incorporated by reference.

In some embodiments, the manual insufflator device may also include one or more barriers to prevent effluent or distention media from migrating from one location to another. The one or more barriers may be positioned at one or more sites, including, but not limited to, any conduit between the insertable member and the area to be protected from contact with the subject's effluent. In some embodiments, the one or more barriers may be disposed between a effluent collection reservoir and an area that is to be protected. FIG. 4 illustrates a barrier in the form of a filter 80 being disposed between the effluent collection reservoir 60 and the pumping member 12. Also, the barriers may be positioned in various locations in order to prevent the media or expelled effluent from inadvertently migrating through the cavity opening and into an open clinical area or the reusable part of the device.

In one alternative embodiment, the barrier 80 may comprise one or more layers of material impervious to the passage of liquids, solids, and particulates, but not gas. Such a barrier is designed to materially reduce the transfer of pathogens, such as viruses and bacteria, mucous and bodily fluids. In one alternative embodiment, the effluent barrier 80 may comprises a hydrophobic membrane to provide an anti-viral and anti-bacterial barrier, including but not limited to a 0.1 micron hydrophobic membrane.

The effluent barrier may also comprise any other well known, commercially available filtration media system impervious to biological matter. The filtration media's performance may be enhanced by an in-line check valve or unidirectional valve on a side of the filtration media. Additionally, the effluent barrier need not be an independent, stand-alone structure. It can form an integral part of any component of the present invention. For example, an appropriate hydrophobic membrane may form an integral part of one or more openings of the effluent reservoir. The barrier may also form an integral part of the interior of a conduit or insertable member.

In another alternative embodiment, the present invention may have an adjustable barrier for limiting or preventing the effluent or media from migrating from one location to another during or after the medical or diagnostic procedure. The adjustable barrier may include, but is not limited to, a clamp, valve, stop clock, slide clamp, or pinch clamp. In this regard, FIGS. 4a and 4b illustrate a clamp 82 in the form of a slide clamp attached to conduit 40 between the insertable member 20 and the effluent collection reservoir 60. Clamp 82 may be used to prevent unwanted flow of effluent and/or media. It should be recognized that clamp 82 may be disposed at any location on the device where it is desirable to block the flow of media. In one alternative embodiment, the insertable member may support a locking pinch clamp.

As discussed in greater detail below, the manual insufflator device may comprise a variety of embodiments. In some embodiments, the insufflator device may be packaged preassembled, or alternatively, unassembled in the form of a kit that can be assembled by the user. In some embodiments, the insufflator device may comprise a consumable portion that is disposed of after use, and a reusable portion that may be repeatedly used in combination with the consumable portion. In some embodiments, the manual insufflator device may function as a manually actuated booster pump that can be used in conjunction with a conventional electro-pneumatic insufflator system.

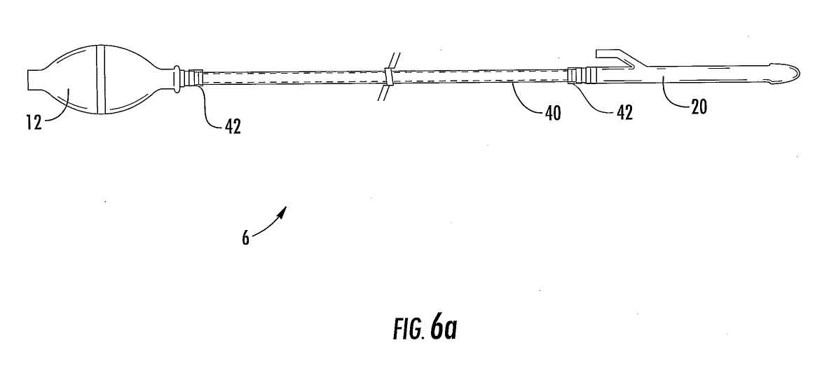

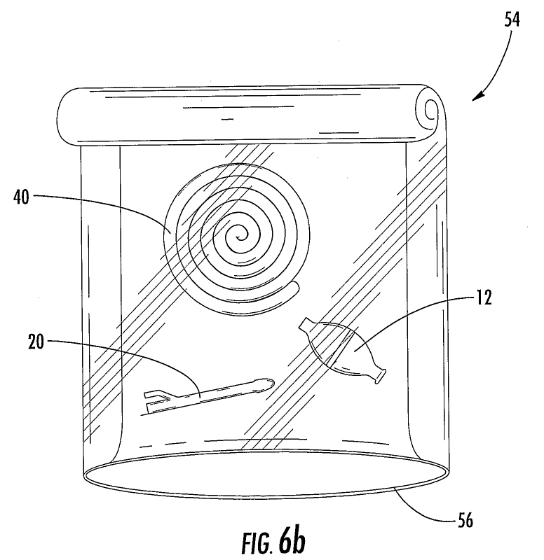

In one alternative embodiment, the invention comprises a consumable manual insufflator device that may be disposed of after use. The manual insufflator device may be available pre-assembled or in the form of an unassembled kit that can be assembled by the user. In one embodiment, manual insufflator device comprises a kit comprising a consumable manual insufflator device that can be disposed of after use. In this regard, FIG. 6a illustrates a manual insufflator device comprising an insertable member 20, pumping member 12, and fluid conduit 40. In this embodiment, the reservoir comprises the surrounding atmosphere from which air may be drawn as the distention media. The individual members may be assembled by the user and may be disposed of after use. FIG. 6b depicts an alternative embodiment of the manual insufflator device 6 that may be available as a kit 54. The kit 54 may comprise the various components of the manual insufflator device disposed in a package 56 in an unassembled state. The kit may be packaged in a variety of containers including, but not limited to, polymeric or plastic bag structures, box structures, polymeric form tray/lid combination structures, and the like.

In one alternative embodiment, the kit 54 may comprise pumping member 12, insertable member 20, and conduit 40. The kit 54 in some embodiments may further comprise various connectors and barriers 82, such as slide or pinch clamp.

In another embodiment, which is discussed in greater detail below, the kit may also comprise a media distention reservoir in the form of an expandable, collapsible, bag-like structure, and means for filling the reservoir with distention media, such as effervescent granules or a disposable CO₂ cartridge. In another embodiment, the kit may comprise a media distention reservoir having an access inlet (see FIG. 1, reference number 11), through which, for example, effervescent granules or a supply tube connected to a CO₂ canister may be inserted.

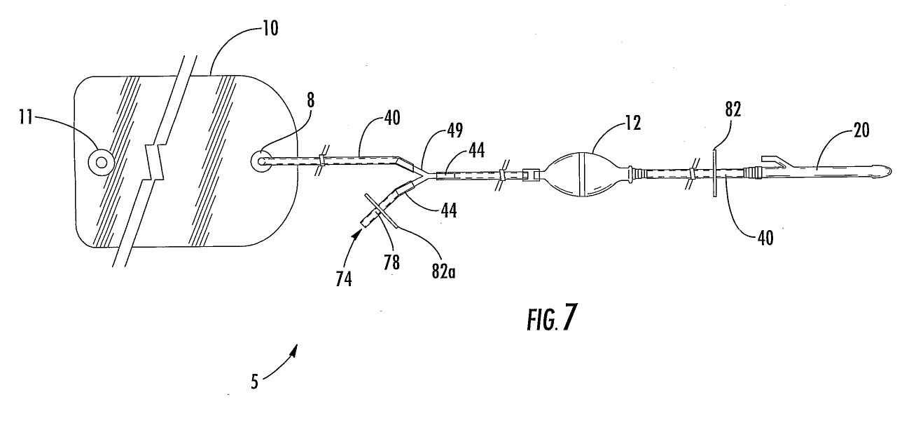

With reference to FIGS. 1 and 7, alternative embodiments of consumable manual insufflator devices are illustrated. These manual insufflator devices may also be available preassembled, or alternatively, in the form of an unassembled kit. Both of these embodiments comprise an insertable member 20, pumping member 12, media distention reservoir 10, and various fluid conduits 40. As shown in FIG. 7, the manual insufflator device 5 in some embodiments may further comprise a 3-way connector 49, such as a Y- or T-connector, adapted for drawing distention media from the surrounding atmosphere. The 3-way connector may define a first fluid pathway between the distention media reservoir 10 and the insertable member 20, and a second fluid pathway between the surrounding atmosphere at 74 and the insertable member 20. A short conduit or tube 78 may be connected to the 3-way connector 49 at one connection end 44 and open to the atmosphere of another. A clamp 82a, such as sliding or pinch clamp, may be sealably positioned on the conduit 78 to prevent egress of distention media through opening 77, thereby maintaining a closed system so that distention media contained in the reservoir may be displaced into the subject's body cavity. If the volume of the distention media is consumed, the user may open clamp 82a on conduit 78. Air from the surrounding atmosphere can then be used to make up for any shortfall of distention media. The 3-way connector 49 and conduit 78 may also be used as an alternative means through which the distention media reservoir may be filled with distention media. For example, an external canister containing distention media, such as CO₂, can be connected to conduit 78 to provide distention media to the reservoir. After the reservoir 10 has been filled, clamp 82a may be closed.

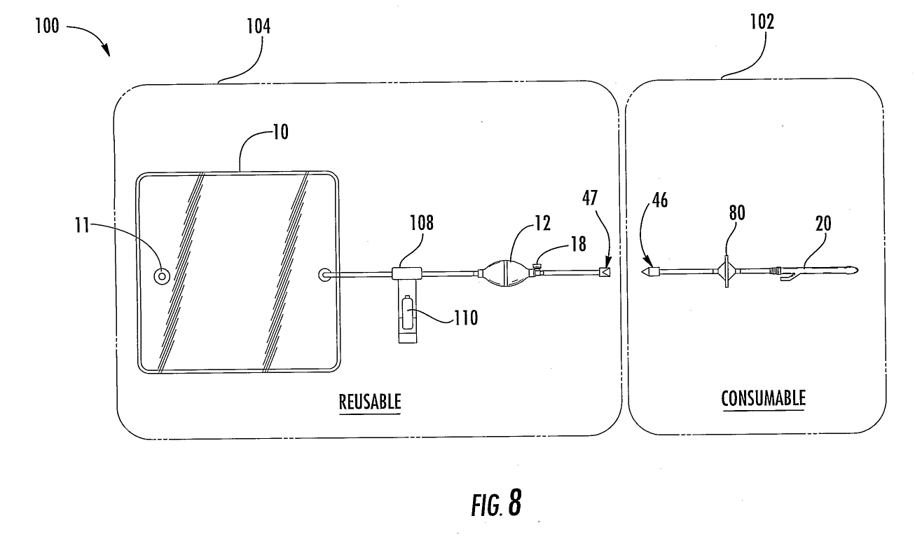

With reference to FIG. 8, an alternative embodiment of the manual insufflator device is illustrated and broadly designated as reference number 100. Manual insufflator device 100 comprises a consumable portion 102 and a reusable portion 104. The removable or disposable nature of the consumable portion 102 assists in preventing contamination of the reuseable portion 104 and cross-contamination of patients. Additionally, it may provide a means of cost-savings because components such as the distention media reservoir and the pumping member may in some embodiments be repeatedly reused.

In one alternative embodiment, the consumable portion 102 may comprise an insertable member 20, effluent barrier 80, and a connectivity means (46, 47) for joining the consumable and reusable portion together. In some embodiments, such connectivity means may include fittings that a user may connect, disconnect, and reconnect as needed. In one alternative embodiment, the connectivity means may include, for example, Colder Products® fittings, Luer fittings, barbed fittings, and the like, and combinations thereof. The reusable portion may comprise a pumping member 12, distention media reservoir 10, and connectivity means. In the alternative embodiment illustrated in FIG. 8, the reusable portion 104 may comprise a CO₂ cartridge connection 108 that is adapted for supplying the reservoir with CO₂ that may be supplied from a disposable CO₂ cartridge 110. Typically, the CO₂ cartridge 110 is a consumable item that may be packaged and sold with the consumable portion 102 of the insufflator device. Alternatively, the distention media reservoir may be filled with a suitable distention media, such as CO₂, in other ways, such as filling the reservoir with CO₂ gas from an external CO₂ canister or by placing effervescent granules into the reservoir. Additionally, in some embodiments, the reusable portion may also include a 3-way connector (not illustrated) that could be used to fill the reservoir 10 from an external canister as described above.

In some embodiments, the barrier 80 may comprise a hydrophobic membrane or equivalent filter media to provide an anti-viral and anti-bacterial barrier so that the reusable portion 104 is not contaminated. In this embodiment, the consumable portion 102 may be available preassembled for easy installation with the reusable portion. After each use, the consumable portion may be disposed of in any suitable manner. Alternatively, the consumable portion may be available unassembled in the form of a kit.

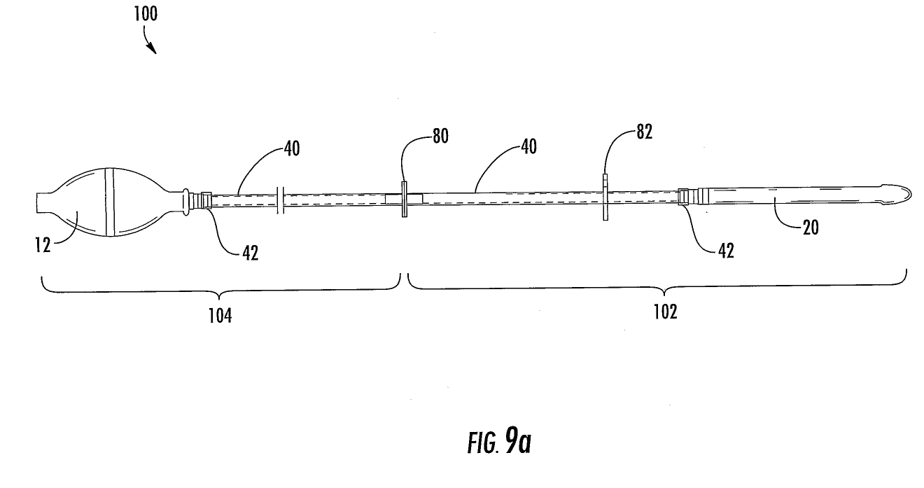

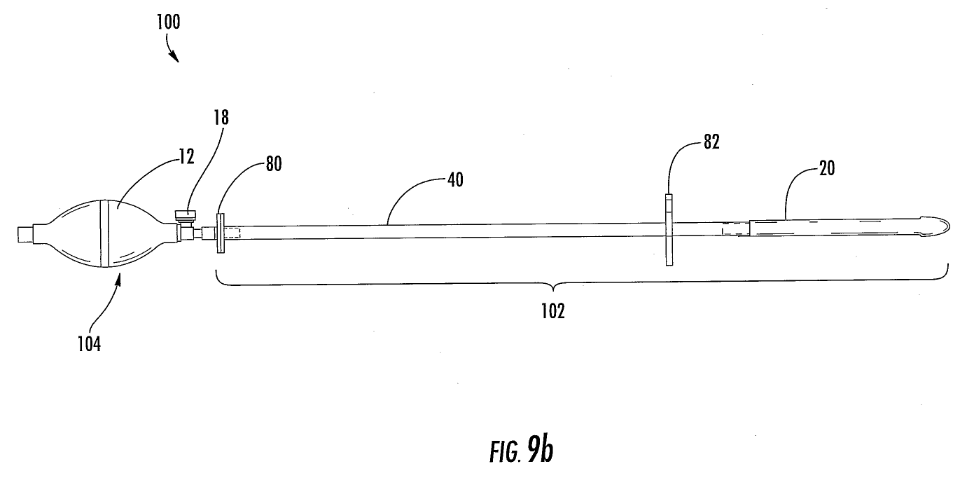

With reference to FIGS. 9a and 9b, two alternative embodiments of insufflator device 100 are illustrated. As shown, the reusable portion may comprise a pumping member 12 that may be adapted to draw distention media directly from the surrounding atmosphere. The consumable portion 102 may comprise an insertable member 20, flow conduit 40, and a hydrophobic membrane 80 or equivalent filter to prevent contamination of the reusable portion. In some embodiments, the consumable portion may be sold as a kit that can be assembled by the user. In other embodiments the consumable portion can be sold preassembled.

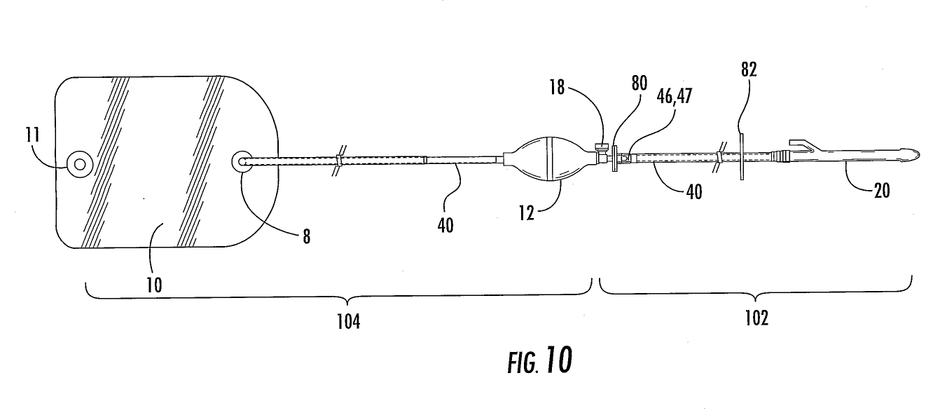

Referring back to FIG. 4a, and to FIG. 10, two additional alternative embodiments of the manual insufflator device 100 are illustrated. FIG. 4a illustrates an embodiment wherein the consumable portion further comprises an effluent reservoir 60. As discussed above, the effluent reservoir 60 traps effluent and helps prevent it from obstructing the flow of distention media from the distention media reservoir 10 to the subject's body cavity. FIG. 10 illustrates an embodiment, wherein the reusable portion 104 may include the reservoir 10 and the pumping member 12. The consumable portion 102 may include the insertable member 20 and a barrier filter 80.

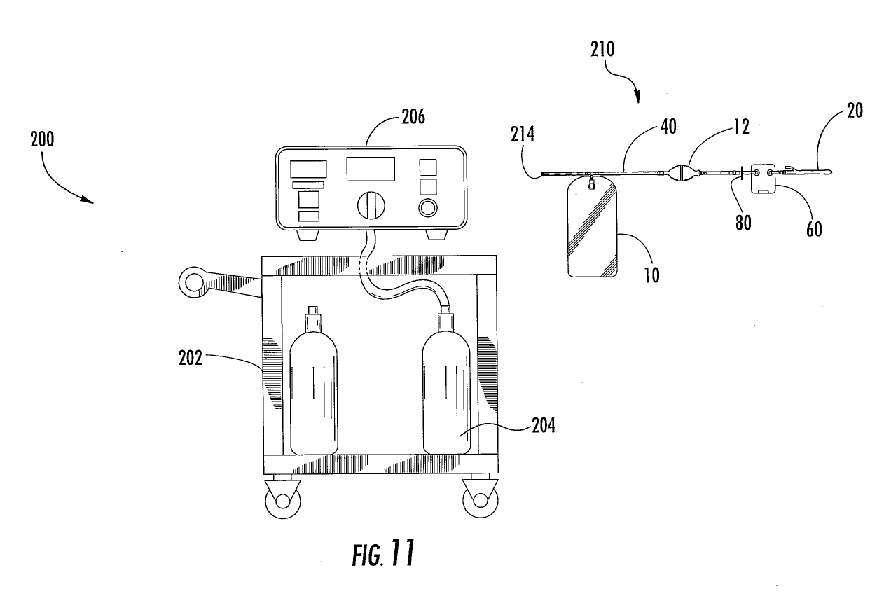

In another alternate embodiment, the manual insufflator device may be used as a manually operated booster in conjunction with a conventional electro-pneumatic insufflator system. A suitable insufflation unit for use with the manual insufflator device of the present invention includes, but is not limited to the E-Z-EM PROTOCO₂L™ Colon Insufflator or a similar device. This product is available from E-Z-EM, Inc., of Westbury, N.Y. In this regard, FIG. 11 illustrates an insufflation system 200 for distending a subject's body cavity comprising an insufflation unit 206 and an administration set 210 that is in accordance with the invention. As shown, the administration set comprises a manually operated insufflator device 210 that may be used to augment the supply of distention media provided by insufflation unit 206.

In one embodiment, insufflator init 206 may be an automatic insufflator unit. Automatic insufflator units suitable for use herein include, but are not limited to, any electronic device for displacing gas into the colon. In one embodiment, the unit is an electro-pneumatic carbon dioxide insufflator, such that the unit delivers CO₂ to the patient's colon for distention by specifying the following parameters at the control interface. When rectally inserting the enema tip of the disposable tube set in a patient, an appropriate distention pressure of CO₂ may include from about 0 to 25 mm Hg. Set flow rates of CO₂ may include about 1-20 L/min, and set pressure from about 10 mm Hg to about 50 mm Hg, preferably about 3 to 6 L/min, and 20-30 mm Hg, respectively.

This insufflator unit 206 may comprise a software controlled electromechanical system that precisely regulates pressure and meters flow of CO₂ from a supply cylinder 204 to the patient. In some embodiments, the manually operated insufflator device 210 may comprise vinyl tubing, two balloon inflators, plastic tubing clamp 82, Flexi-Tip 20 with Flexi-Cuff silicone elastomer retention cuff, 0.1 micrometer hydrophobic filter 80, 100 mL effluent collection container 60, distention media reservoir 10, pumping member 12, and connector 214 to PROTOCO₂L Colon Insufflator. The system may be provided in latex free form to prevent allergic reaction of the patient.

In one embodiment, the system 200 includes cart 202, which may be designed to accommodate the human factors associated with the environment in which the present invention may be used. Its primary functions are support of the insufflator unit and CO₂ supply cylinders 204 on a mobile platform within the CT or colonoscopy suite. Additionally, cart 202 may provide a mounting fixture for the manually operated insufflator device 210 and associated components such as an effluent trap so that it is kept upright during the procedure. This may maximize its effectiveness by localizing any expelled liquid effluent/stool at the bottom of the trap away from the gas lumen. Also, the vertical height of the effluent reservoir may be kept below the insufflator and exam table. Fixation of the effluent reservoir at a position lower than both the insufflator and individual facilitates the collection of effluent into the reservoir through gravity.

With reference to FIG. 12, a manual insufflator device that may be used in conjunction with an electro-pneumatic insufflation unit 206 is illustrated and broadly designated as reference number 210a. As shown, the manual insufflator device may comprise an insertable member 20, pumping member 12, and a distention media reservoir 10. During use, the manual insufflator device 210a may be connected to the insufflation unit outlet via connection 214. Distention media is provided by the insufflation unit and inflates the distention media reservoir 10 which is in fluid communication with conduit 40 through T-connector 212. If desired, the operator can manually override distention of the insufflation unit by applying pumping pressure to pumping member. As a result, the manually operated insufflation device 210a may function as a distention booster for optimizing distention of the subject's body cavity. As shown, in some embodiments the manual insufflation device may comprise a consumable portion and a reusable portion. The device 210a may also comprise an effluent reservoir 60 and various filters 80 and barriers 82 to prevent the unwanted flow of distention media and/or effluent.

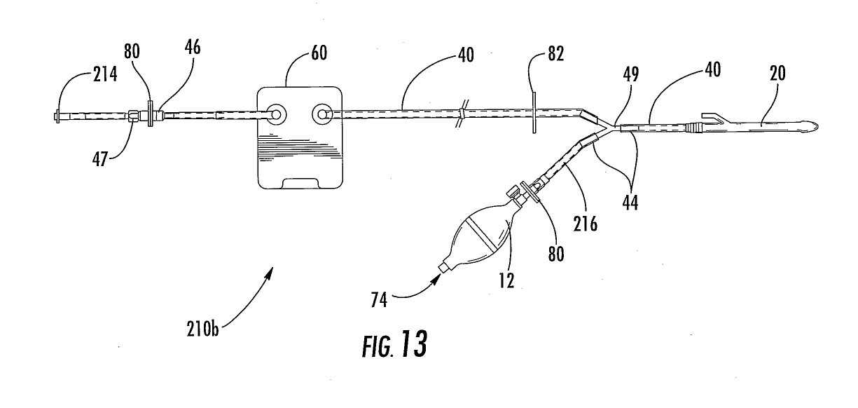

In an alternate embodiment, the manual insufflator device can provide additional distention media in the form of air from the surrounding atmosphere. In this regard, FIG. 13 illustrates a manual insufflator device 210b for drawing air directly from the surrounding atmosphere. As shown, manual insufflator device 210b may comprise a 3-way connector 49 connected to both conduits 40 to define a first fluid pathway from the insufflation unit to the insertable member 20, and a second fluid pathway from the atmosphere, through pumping member 12, and to insertable member 20. The pumping member 12 is adapted for drawing air directly from the atmosphere. The manual insufflator device 210b may be connected to the insufflation unit at connector 214. During operation, distention media is provided by the insufflation unit through conduit 40. Pumping member 12 is adapted to be in fluid communication with the insertable member 20 through 3-way connector 49. If desired, the operator may draw air from the atmosphere by applying manual pressure to pumping member 12. Manual insufflator device 210b may also comprise a reusable portion and a consumable portion. As shown, barrier filters 80 may be present to help prevent effluent from contaminating the pumping member 12 and from traveling pass connector 214 in the direction of the insufflation unit. In some embodiments, the manual insufflator device 210b may also comprise an effluent reservoir 60.

Other modifications and other embodiments of the invention set forth herein will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

Further, throughout the description, where compositions are described as having, including, or comprising specific components, or where processes or methods are described as having, including, or comprising specific steps, it is contemplated that compositions of the present invention may also consist essentially of, or consist of the recited components, and that the processes or methods of the present invention also consist essentially of or consist of the recited steps. Further, it should be understood that the order of steps or order for performing certain actions are immaterial so long as the invention remains operable. Moreover, two or more steps or actions may be conducted simultaneously with respect to the invention disclosed herein.

That which is claimed:

1. A device for manually distending a subject's body cavity comprising:
a) a hollow insertable member adapted for insertion through an opening of a body cavity;
b) a distention media reservoir adapted for releasably confining distention media therein, the distention media reservoir in fluid communication with the insertable member; and
c) a manually operable pumping member adapted for introducing distention media from the distention media reservoir, through the insertable member, and into a body cavity of a subject, whereby the body cavity is distended.

2. A device according to claim 1, further comprising a distention media disposed in the distention media reservoir.

3. A device according to claim 2, wherein the distention media comprises CO₂ gas.

4. A device according to claim 1, wherein the distention media reservoir comprises an expandable, collapsible bag.

5. A device according to claim 1, wherein the media distention reservoir further comprises an access inlet for receiving distention media therein.

6. A device according to claim 1, wherein the pumping member comprises a hollow structure having a bulb, ball, ellipsoidal, or spherical shape.

7. A device according to claim 1, wherein the pumping member comprises a flexible elastomeric material.

8. A device according to claim 1, further comprising a valve disposed between the pumping member and the insertable member, and that is capable of relieving pressure, venting distention media, or combinations thereof.

9. A device according to claim 1, further comprising a 3-way connector disposed between the distention media reservoir and the pumping member, the 3-way connector defining a first fluid pathway between the distention media reservoir and the body cavity, and a second fluid pathway between the surrounding atmosphere and the body cavity, whereby air is displaced from the atmosphere and introduced into the body cavity.

10. A device according to claim 9, wherein the second fluid pathway further comprises a conduit disposed on the connection of the 3-way connector that is adapted for fluid communication with the atmosphere, and wherein the conduit comprises one or more barriers disposed on the conduit, the barriers adapted to prevent distention media from escaping the insufflator device.

11. A device according to claim 1, further comprising a first conduit disposed between the distention media reservoir and the pumping member, and a second conduit disposed between the pumping member and the insertable member, wherein the first and second members define a fluid pathway from the distention media reservoir to the insertable member.

12. A device according to claim 1, further comprising one or more barriers disposed on the first or second conduit, the barriers adapted to prevent effluent from migrating to an undesired location.

13. A device according to claim 12, wherein the one or more barriers comprise an anti-viral or anti-bacterial barrier.

14. A device according to claim 13, wherein the one or more barriers comprises a hydrophobic filter.

15. A device according to claim 14, wherein the one or more barriers comprises a 0.1 micrometer hydrophobic membrane.

16. A device according to claim 12, wherein the one or more barriers comprises a fastening clamp, clip, snap lock, pinch clamp, or any equivalent thereof.

17. A device according to claim 1, further comprising an effluent reservoir disposed between the pumping member and the insertable member, the reservoir having multiple openings leading into the interior of the reservoir and defining a fluid pathway between the pumping member and the insertable member, the multiple openings adapted to receive a bidirectional flow of medium and effluent into or through the reservoir.

18. A device according to claim 17, wherein the effluent reservoir comprises a flexible, collapsible bag.

19. A device according to claim 17, wherein the effluent reservoir comprises a substantially rigid container.

20. A device according to claim 17, further comprising one or more barriers disposed on the second conduit between the pumping member and the effluent reservoir, the barriers adapted to prevent effluent from migrating to an desired location.

21. A device for use in conjunction with a medical or diagnostic apparatus comprising:
a. a first conduit having a proximal and distal end, the first conduit adapted for interfacing with one or more medical or diagnostic apparatus;
b. a distention media reservoir adapted for releasably confining distention media therein, the distention media reservoir having an access connection interfaced with the first conduit,
c. a pumping member adapted for displacing distention media from the distention media reservoir, the pumping member having an inlet for receiving distention media and an outlet for expelling distention media, the inlet interfaced with the proximal end of the first conduit;
d. a second conduit having a proximal end and a distal end, the distal end interfaced with the outlet of the pumping member; and
e. an insertable member adapted for insertion into the body cavity of a patient, the insertable member having a front portion and a rear portion, including at least one hollow area extending from the front portion to the rear portion, the rear portion interfaced with the proximal end of the second conduit, whereby the body cavity of the subject is distended by introducing distention media therein.

22. The device according to claim 21, wherein the interface between the access outlet and the first conduit comprises a T-connector.

23. The device according to claim 21, wherein the media distention reservoir contains distention media comprising CO₂ gas disposed therein.

24. The device according to claim 21, further comprising at least one connection means for attaching the first conduit to the distention media reservoir, the at least one connection means including Luer connection, Colder connection, barbed connection, or male/female connection.

25. The device according to claim 21, wherein the distal end of the first conduit is interfaced with an electro-pneumatic insufflator apparatus.

26. The device according to claim 21 further comprising an effluent reservoir disposed between the pumping member and the insertable member.

27. The device according to claim 21, further comprising one or more barriers disposed on the second conduit between the pumping member and the effluent reservoir, the one or more barriers adapted to prevent effluent from migrating to a desired location.

28. The device according to claim 27, wherein the one or more barriers comprises a 0.1 micrometer hydrophobic membrane.

29. The device according to claim 21, wherein the front portion of the insertable member comprises a tip structure supported thereon, the tip structure adapted for initiating entry of the insertable member into an opening to the body cavity.

30. The device according to claim 29, wherein the insertable member has a shaft with an inner and outer surface, and wherein an inflatable balloon is positioned on the outer surface of the shaft for inflation from a substantially flat configuration into a distended configuration around the shaft upon insertion of the tip structure into a body cavity.

31. The device according to claim 30, wherein the balloon is inflated to prevent discharge of effluent from a body cavity.

32. The device according to claim 21, further comprising a valve disposed between the pumping member and the insertable member, and that is capable of relieving pressure, venting distention media, or combinations thereof.

33. A manually operated device for introducing a fluid into the body cavity of a subject comprising:
a reusable portion adapted to be in fluid communication with a consumable portion,
the reusable portion comprising a distention media reservoir in fluid communication with a manually operated pumping member, and a first connector adapted for connecting the reusable portion to the consumable portion,
the consumable portion comprising an insertable member adapted for fluid communication with the distention media reservoir, at least one barrier adapted to prevent effluent from migrating to an undesired location, and a second connector adapted for releasably connecting the consumable portion to the first connector whereby the consumable portion is separable from the reusable portion.

34. The device according to claim 33, further comprising a first conduit defining a fluid pathway from the distention media reservoir through the pumping member, to the first connector, and a second conduit defining a fluid pathway from the second connector to the insertable member, whereby distention media disposed in the distention media reservoir is introduced into the body cavity of a subject.

35. The device according to claim 33, wherein the distention media reservoir comprises the surrounding atmosphere.

36. The device according to claim 33, wherein the at least one barriers comprise an anti-viral or anti-bacterial barrier.

37. A device according to claim 36, wherein the at least one barriers comprises a hydrophobic filter.

38. The device according to claim 33, wherein the media distention reservoir comprises an interior for releasably confining distention media therein, and an inlet access for receiving distention media into the interior.

39. The device according to claim 33, wherein the distention media reservoir comprises a flexible, collapsible bag.

40. The device according to claim 33, wherein the reusable portion further comprises a 3-way connector disposed between the distention media reservoir and the pumping member, the 3-way connector defining a first fluid pathway between the distention media reservoir and the body cavity, and a second fluid pathway between the surrounding atmosphere and the body cavity, whereby air is displaced from the atmosphere and introduced into the body cavity.

41. The device according to claim 40, wherein the second fluid pathway further comprises a conduit disposed on the connection of the 3-way connector that is adapted for fluid communication with the atmosphere, and wherein the conduit comprises one or more barriers disposed on the conduit, the barriers adapted to prevent distention media from escaping the insufflator device.

42. The device according to claim 33, wherein the consumable portion further comprises an effluent reservoir disposed between the insertable member and the at least one barrier.

43. The device according to claim 33, further comprising a valve disposed between the pumping member and the insertable member, and that is capable of relieving pressure, venting distention media, or combinations thereof.

44. A kit for use in medical procedures comprising:
an insertable member adapted for insertion into a body cavity of a patient;
a pumping member adapted for displacing air from the surrounding atmosphere and introducing the air into the body cavity; and
a conduit adapted to be in fluid connection with the insertable member and the pumping member whereby the conduit defines a fluid pathway between the pumping member and the insertable member.

45. The kit according to claim 44, wherein the pumping member, insertable member, and the conduit are disposed in a container.

46. The kit according to claim 45, further comprising connector means for attaching one end of the conduit to the pumping member, and the other end to the insertable member.

47. The kit according to claim 46, wherein the connector means comprises friction fit connector, Luer connector, Colder Products® connection, barbed connector, or male/female connector.

48. The kit according to claim 44, wherein the pumping member comprises a hollow structure having a bulb, ball, ellipsoidal, or spherical shape, the pumping member formed of a flexible elastomeric material.

49. The kit according to claim 48, wherein the pumping member comprises an inlet for receiving air into the hollow structure, and an outlet for expelling air from the hollow structure.

50. The kit according to claim 44, further comprising relief valve adapted to be disposed between the pumping member and the insertable member, and wherein the valve is capable of relieving pressure, venting distention media, or combinations thereof.

51. A kit for use in medical procedures involving the distention of a patient's body cavity, the kit comprising:
an expandable, collapsible pouch having an interior adapted for releasably confining a distention media;
an insertable member adapted to be in flow communication with the expandable pouch and a body cavity of a patient;
a means for filling the expandable pouch with distention media; and
a pumping member adapted for displacing distention media from the expandable pouch, through the insertable member, and into the body cavity.

52. A kit according to claim 51, further comprising a first conduit adapted to be disposed between the expandable pouch and the pumping member, and a second conduit adapted to be disposed between the pumping member and the insertable member.

53. A kit according to claim 51, wherein the first and second conduits comprise connection means for attaching the first conduit at one end to the distention media reservoir and at the other end to the pumping member, and attaching the second conduit at one end to pumping member and at the other end to the pumping member, the at least one connection means including Luer connection, Colder Products® connection, barbed connection, or male/female connection.

54. A kit according to claim 51, wherein filling means comprises a disposable CO₂ cartridge, a compound capable of forming a gas upon mixing with water, dry ice, or an external CO₂ canister adapted for fluid communication with the media distention reservoir.

55. A kit according to claim 51, wherein the expandable pouch comprises a flexible thermoplastic material.

56. A kit according to claim 51, further comprising a 3-way connector adapted to be disposed between the pouch and the pumping member, the 3-way connector defining a first fluid pathway between the pouch and the body cavity, and a second fluid pathway between the surrounding atmosphere and the body cavity, whereby air is displaced from the atmosphere and introduced into the body cavity.

57. A kit according to claim 52, further comprising one or more barriers disposed on the first or second conduit, the barriers adapted to prevent effluent from migrating to a desired location.

58. A kit according to claim 57, wherein the one or more barriers comprise an anti-viral or anti-bacterial barrier.

59. A kit according to claim 58, wherein the one or more barriers comprises a 0.1 micrometer hydrophobic membrane.

60. A kit according to claim 51, wherein the distention media reservoir includes an access inlet adapted for receiving distention media into an interior space of the distention media reservoir.

61. A kit according to claim 60, wherein the distention media reservoir is adapted to be filled with distention media from an external CO₂ canister.

62. A kit according to claim 51, further comprising an effluent reservoir disposed between the insertable member and the pumping member.

63. A system for performing a medical or diagnostic procedure on a subject comprising:
a) an electro-pneumatic insufflation unit adapted for introducing a distention media into a body cavity of a subject, the unit having a distention media outlet; and
b) a manually operated insufflator device in fluid communication with the distention media outlet, the insufflator device comprising:
a first conduit having a proximal and distal end, the proximal end interfaced with the distention media outlet;
a distention media reservoir interfaced with the first conduit;
a pumping member having an inlet and an outlet, the inlet being interfaced with the proximal end of the first conduit;
a second conduit having a proximal and distal end, the distal end interfaced with the pumping member outlet; and
an insertable member adapted for introducing distention media into the body cavity, the insertable member interfaced with the proximal end of the second conduit, such that a first fluid pathway from the insufflation unit;

64. A system according to claim 63, wherein the distention media provided by the insufflation unit comprises CO₂ gas.

65. A system according to claim 64, wherein the interface between the distention media reservoir and the first conduit comprises a T-connector, and the distention media reservoir is disposed between the distention media outlet and the pumping member, whereby distention media may be provided by the insufflation unit and the distention media reservoir.

66. A system according to claim 65, wherein the distention media reservoir comprises a flexible, collapsible bag.

67. A system according to claim 65, wherein the insufflation unit supplies the distention media reservoir with distention reservoir via the T-connector.

68. A system according to claim 63, wherein the distention media reservoir comprises the atmosphere, and the interface between the distention media reservoir and the first conduit comprises a 3-way connector, the 3-way connector defining a first fluid pathway between the distention media outlet and the body cavity, and a second fluid pathway between the surrounding atmosphere and the body cavity, whereby air is displaced from the atmosphere and introduced into the body cavity.

69. A system according to claim 63, further comprising an effluent reservoir disposed between the pumping member and the insertable member, the reservoir having multiple openings leading into the interior of the reservoir and defining a fluid pathway between the pumping member and the insertable member, the multiple openings adapted to receive a bidirectional flow of medium and effluent into or through the reservoir.

70. A system according to claim 69, further comprising one or more barriers disposed on the second conduit between the effluent trap and the pumping member, and wherein the one or more barriers comprises a 0.1 micrometer hydrophobic membrane.

71. A system according to claim 63, further comprising one or more barriers disposed on the first or second conduits, the barriers adapted to prevent effluent from migrating to a desired location.

72. A system according to claim 71, wherein the one or more barriers comprise an anti-viral or anti-bacterial barrier.

73. A system according to claim 72, wherein the one or more barriers comprises a hydrophobic filter.

74. A system according to claim 73, wherein the one or more barriers comprises a 0.1 micrometer hydrophobic membrane.

75. A system according to claim 71, wherein the one or more barriers comprises a fastening clamp, clip, snap lock, pinch clamp, or any equivalent thereof.

76. A system according to claim 70, wherein the second conduit further comprises a connector means defining a reusable portion and a consumable portion, the reusable portion comprising the pumping member, the first conduit, and the distention media reservoir, and the consumable portion comprising the insertable member, the effluent trap, the one or more barriers disposed on the second conduit, and at least a portion the second conduit, and wherein the connector means is adapted for releasably attaching the consumable portion to the reusable portion.

77. A system according to claim 63, further comprising a valve disposed between the pumping member and the insertable member, and that is capable of relieving pressure, venting distention media, or combinations thereof.

78. A method of distending a body cavity comprising the steps of:
a) providing the manual insufflator device of claim 1;
b) inserting the insertable member into the body cavity of a subject;
c) displacing the distention media from the media distention reservoir;
d) introducing distention media into the body cavity.

79. A method according to claim 78, further comprising the step of filling an interior of the distention media reservoir with distention media.

80. A method according to claim 79, wherein the step of filling the distention media reservoir includes at least one of the following steps:
mixing effervescent granules with water in the interior of the media distention reservoir;
placing dry ice into the interior of the media distention reservoir; and
inserting a supply tube of a CO₂ canister into the interior of the media distention reservoir, and supplying CO₂ gas from the canister.

81. A method according to claim 80, wherein the step of introducing distention media further comprises the step of distending the body cavity using air.

82. A method according to claim 78, wherein the step of displacing distention media further comprises applying manual pressure to the pumping member.

83. A method according to claim 78, further comprising the step of connecting the manual insufflator device to an electro-pneumatic insufflation unit, whereby a fluid pathway from the insufflation unit to the insertable member is defined.

84. A method according to claim 83, further comprising the step of distending a subject's body cavity using the device of claim 1 in conjunction with the insufflation unit.

85. A method according to claim 78, further comprising the step of imaging the subject's body cavity.

86. A method according to claim 85, wherein the step of imaging further comprises scanning the body cavity with using CT.
